(19) United States
(12) Patent Application Publication
(10) Pub. No.: US 2009/0304576 A1
(43) Pub. Date: Dec. 10, 2009

(54) DEVICE FOR DELIVERY OF ANTI-CANCER AGENTS TO TISSUE

(76) Inventors: Stephen L. Warren, Fort Collins, CO (US); Gregory A. Gould, Wrentham, MA (US); Kevin Lillehei, Englewood, CO (US)

Correspondence Address: SCHWEGMAN, LUNDBERG & WOESSNER, P.A., P.O. BOX 2938, MINNEAPOLIS, MN, 55402, US

(21) Appl. No.: 12/375,349

(22) PCT Filed: Jul. 6, 2007
(86) PCT No.: PCT/US07/15549

§ 371 Date: Aug. 26, 2009

Related U.S. Application Data
(60) Provisional application No. 60/821,775, filed on Aug. 8, 2006.

(51) Int. Cl. A61K 51/12; A61K 9/00; A61P 35/00; A61K 49/06

(52) U.S. Cl. 424/1.11; 424/423; 424/426; 424/9.3

(57) ABSTRACT

A filament comprising a biocompatible material and a bioactive agent, adapted for implantation within the tissue of a patient wherein the bioactive agent is released over a period of time, is provided. An array of a plurality of the filaments implanted within the tissue of a patient, an array assembler, and a matrix comprising a plurality of filaments and a base adapted for implantation within the tissue of a patient, are further provided. A method for treatment of a malcondition in a patient comprises implantation of a filament, an array of filaments, or a matrix. The bioactive agent can be a chemotherapeutic agent or a radiotherapeutic agent. A radiotherapeutic agent is 123I- or 125I-IUDR, for example in treatment of an advanced stage localized brain tumor such as glioblastoma multiforme.

### CLAIM OF PRIORITY

The application claims the priority of U.S. Provisional Application Ser. No. 60/821,775, filed Aug. 8, 2006, which is incorporated herein by reference in its entirety.

### BACKGROUND

In the treatment of neoplasia such as solid tumors, particularly in the early stages, surgical excision or ablation often provides a successful form of therapy. Often, surgery is accompanied with chemotherapy, radiotherapy, or a combination of adjuvant therapies designed to eliminate malignant cells not removed by the surgery. However, when a neoplasm is more advanced, such as in the case of advanced stage but still localized (not metastasized) solid tumors, surgery and conventional adjuvant treatments like systemic chemotherapy or external beam radiation are less effective.

Locally advanced or locally invasive solid tumors are primary cancers that have not spread to distant sites, but have extensively invaded or infiltrated into the otherwise healthy tissues surrounding the tumor. Locally advanced tumors are found in tissues throughout the body. Unlike early stage tumors, they may not be amenable to complete surgical excision, due to the invasion of the surrounding tissues by tumor processes. Any surgical procedure that would serve to remove all the cancerous cells would also be likely to maim or destroy the organ in which the cancer originated and would necessarily result in extensive destruction of healthy tissue.

In cases involving locally advanced tumors, surgery may be used for gross excision, referred to as “debulking,” but the surgeon at present does not have the tools to eliminate individual tumor cells, microscopic tumor processes, or tumor-associated vasculature from the normal tissue surrounding the tumor excision site. It is often critical to minimize the volume of surrounding tissue that is excised in such operations, for example in the case of tumors of the central nervous system, due to the damage to normal function that occurs as a result of tissue loss. Thus, in such cases surgery is accompanied by radiation therapy and systemic chemotherapy in an attempt to kill cancerous cells remaining in the surrounding tissue.

Conventional radiation therapy, using ionizing radiation beams (X-ray, gamma ray, or high energy beta particle), while well-established as an anti-cancer treatment modality, is not curative in the majority of patients whose cancer is still confined to the primary anatomic site or region. Another form of radiation therapy is brachytherapy, the implantation of sealed radioactive sources emitting gamma rays or high energy beta particles within the tissue adjacent to the tumor site, for example in treatment of prostate cancer. For example, see U.S. Pat. Nos. 6,248,057, 6,743,211, and 6,905,455.

However, even with the addition of systemic agents, one third of patients with locally advanced solid tumors relapse (Vijaykumar, S, and Hellman, S., “Advances in Radiation Oncology,” Lancet, 349[S11}: 1-3 (1997)). Furthermore, ionizing radiation, whether from a beam or from an isotopic implant emitting high energy radiation, lacks specificity for tumor cells, and collateral damage to normal tissues cannot be avoided; ionizing radiation is itself oncogenic.

A area of recent interest in radiotherapy involves the use of Auger electron emitters such as ¹²⁵I. Auger electrons are emitted by radionuclides that decay by electron capture and internal conversion, and have energies even lower than the energy of the beta particle emitted by tritium, but have much shorter half-lives and thus much higher specific activities than does tritium. Thus effect is multiplied by the fact that some Auger emitters give off multiple Auger electrons with each nuclear transformation. The low energy of the Auger electrons results in short particle pathlengths in tissue, which is desirable for reducing collateral damage. One molecular entity incorporating ¹²⁵I is iodouridinedeoxyriboside, a thymidine analog that is incorporated into cellular DNA. In this situation, the Auger electrons with their very short range are particularly well-suited to damage the cell in which they are incorporated with minimal effect on the surroundings. For example, see U.S. Pat. No. 5,077,034.

Systemic chemotherapy also suffers from a lack of tumor specificity and the possibility of collateral damage to normal tissues, as the chemotherapeutic agents are distributed throughout the body and exert their effects on normal cells as well as malignant cells. Typically, chemotherapy agents act on cells undergoing DNA synthesis and cell division, and thus may impact many cell populations throughout the body in addition to the target cells.

The deficiencies of current treatment modalities are especially glaring with respect to specific types of cancer. For example, in currently favored courses of treatment of glioblastoma multiforme (GBM), a highly aggressive type of cancer that constitutes the most common form of brain malignancy, surgical resection is accompanied by external beam radiation and administration of oral temozolomide (a prodrug for an alkylating agent). Despite the treatment, it has been reported that the median prolongation of survival is only about 2-3 months. A recent study showed that the overall survival of patients with newly diagnosed GBM was only 42% at 6 months, 18% at one year, and 3% at 2 years (Ohgaki, et al., “Genetic pathways to glioblastoma: A population-based study,” Cancer Research, 64:6892-6899 (2004)).

Recently, techniques have been developed to increase the effective concentration of chemotherapeutic agents at a tumor site. In the treatment of GBM, the technique of interstitial chemotherapy has been used with some success. Implantation of carmustine (a nitrogen mustard alkylating agent) wafers within the brain adjacent to the tumor site has been shown to increase the median survival from 11.6 months to 13.9 months in patients also treated with surgery and radiation beam therapy (Westphal, M., et al., “A phase III trial of local chemotherapy with biodegradable carmustine (BCNU) wafers in patients with primary malignant glioma,” Neuro-oncology, 5:79-88 (2003)). This study also reported that substitution of brachytherapy for radiation beam therapy did not further increase the median survival time. Interstitial chemotherapy may be particularly well suited for treatment of GBM, as greater than 80% of GBM tumors that recur following surgical resection are localized within 2 cm of the surgical margin (Hochberg, F. H., and Pruitt, A., Neurology, 30:907-911 (1980)).

Localizing the concentration of the chemotherapeutic agent by physical techniques (as distinct from biochemical targeting) thus seems to offer certain advantages compared to systemic chemotherapy, as shown by the success enjoyed with carmustine wafer implantation. However, the challenge is great, as the majority of chemical entities do not diffuse far in brain tissue or in many other types of solid tissue. The range of diffusion of molecular entities in solid tissues such as brain is limited. A study of the penetration distance of more than structurally diverse molecules found that the mean distance from the source at which the concentration dropped to less than 10% of the starting concentration was in the order of 0.8 and 2 mm. It was estimated that in the case of carmustine, the maximum effective range from a source was about 5 mm. Compounds of higher molecular weight may in fact penetrate further than compounds of lower molecular weight, due to decreased clearance of the higher molecular weight compounds from the tissue.

Another development in physically localized delivery of chemotherapeutic agents is convection enhanced delivery. In this technique as applied to brain tumors, a fluid is delivered directly to a site in the brain and not through the circulatory system. The fluid is applied under a pressure head such that the liquid moves through the interstices of the tissue, carrying with it any dissolved materials. For example, see Hall, W. A., Rustamzadeh, M.D., and Asher, A. L., “Convection-enhanced delivery in clinical trials,” Neurosurg. Focus, 14(2), 1-4, (2003). Convection enhanced delivery thus serves to increase the effective distance over which a bioactive agent can be delivered in solid tissue.

There is an ongoing need for additional, more effective methods for treatment of cancers such as solid tumors involving the delivery of chemotherapeutic and radiotherapeutic agents to specific sites in tissues. More particularly, there is a need for more effective delivery methods for therapeutic agents at the margins of surgical resections in the treatment of advanced stage localized solid tumors, particularly in the central nervous system.

### SUMMARY

An embodiment of the present invention is directed to a medical device that includes an implantable filament, the filament being composed of a biocompatible, preferably biodegradable and thus bioabsorbable, material and a bioactive agent. The filament, which is linear or curvilinear, includes a tip, a shaft and a root, and is adapted for implantation within a solid tissue of a patient. Upon implantation, the filament releases the bioactive agent into the surrounding tissue over a period of time, ranging from a few days to many weeks, the bioactive agent being therapeutic for a malcondition of the patient. One or more such filaments can be implanted within tissue in the vicinity of a tumor, such as an advanced stage solid tumor that is localized in a tissue such as nervous system tissue. The filaments release the bioactive agent such that the agent is concentrated in the tissue that may contain cancerous cells but which contains sufficient normal tissue to contraindicate surgical removal. The filaments themselves can undergo biodegradation into non-toxic, soluble components, so that removal of the filament after discharge of the bioactive agent is unnecessary.

An embodiment of a filament of the invention, while being of sufficient rigidity and strength to penetrate a type of tissue into which it is adapted to be emplaced, can be adapted to minimize tissue trauma resulting from the penetration. For example, the flexibility and deflectability of a filament of the invention is adapted to avoid penetration of tissue that is particularly susceptible to damage such as blood vessels within brain tissue.

The filament may further comprise an anchor or a plurality of anchors. An anchor is a structure on the tip, shaft, or root of a filament that serves to hold the filament securely in place within the tissue, as well as enabling an increase in the amount of bioactive agent present in the filament by increasing the total volume of the filament. The filament may further comprise a rib or a plurality of ribs that also serve to increase the amount of bioactive agent that is available for release. A rib can be adapted to provide the flexibility and deflectibility that serve to minimize tissue trauma while allowing for penetration of a target tissue or organ. A plurality of anchors, a plurality of ribs, or both, can be present.

A bioactive agent adapted for treatment of a tumor, contained by a filament of the invention for release into tissue, can be a chemotherapeutic agent, such as carmustine, or a radiotherapeutic agent, such as ¹²⁵I-IUDR. More than a single bioactive agent can be included in the filament. The filament can include both a chemotherapeutic agent and a radiotherapeutic agent, or a plurality of either type or of both types.

A filament of the invention can also comprise additional components, such as a radiopaque agent to allow visualization by fluoroscopy, or a radioactive agent for imaging using techniques such as SPECT or PET, or an MRI-active agent to enhance visualization by magnetic resonance imaging.

An embodiment of the invention further concerns an array of filaments, adapted to be disposed within a tissue of a patient in need thereof. The array is preferably a regular array, wherein the filaments are disposed in a parallel or a radial three-dimensional arrangement, and can be evenly spaced. The filaments are preferably spaced closely enough together that the distance between then is no greater than about twice the distance over which the bioactive agent can diffuse in the tissue in sufficient concentration to provide the desired therapeutic effect. The filaments may or may not be affixed to a base, i.e., a structure that holds the filaments in a fixed spatial relationship to each other.

The filaments making up the array may be emplaced within a tissue individually, in subsets of the total number, or all at once. The plurality of filaments can be emplaced within the tissue by using an array template, that is, a guide structure that serves to exactly position each filament or subset of filaments relative to the other filaments but that does not remain in the tissue after the array is emplaced. Alternatively, the plurality of filaments can be affixed to a base (the assembly referred to herein as a “matrix”) such that the entire array is inserted into the tissue simultaneously, wherein the base can remain in the tissue in association with the filament array after emplacement or can be removed from the filament array after emplacement. Insertion of an array, or of a matrix, or both, can take place in conjunction with tumor debulking surgery, following removal of the bulk of the tumor but prior to closing the exposed tissue. The surgeon can emplace arrays of filaments in any combination of the various possible configurations around the site of the excised tumor bulk. Regardless of how the filament array is emplaced, the filaments are preferably disposed sufficiently close to each other within the tissue such that bioactive agent, when released, diffuses to occupy substantially the entire volume of tissue encompassed by the array with a therapeutically effective concentration of the agent.

An embodiment of the invention is directed to a matrix that includes a plurality of implantable filaments incorporating a bioactive agent, and a base. The filaments are affixed to the base, and the base serves to align the plurality of the filaments in a spatially defined array when implanted in solid tissue. The filaments may be permanently affixed to the base, such that the filaments are inserted into the tissue by application of pressure on the base of the matrix such that the filaments concurrently penetrate the tissue, or the filaments may be removable affixed to the base, such that the base may serve as a template for sequential insertion of filaments to form the defined array within the tissue. After insertion, the base may remain in place as part of a matrix, or the base may be removed to leave a filament array. The filaments may be aligned in a parallel manner when emplaced within the tissue, or in an outwardly (positively) or inwardly (negatively) radial manner, or any arrangement as may be therapeutically indicated.

An embodiment of the present invention also provides a method for using a medical device comprising a single filament, an array of a plurality of filaments, or a matrix including an array of a plurality of filaments and a base. The filament or a plurality of filaments or a plurality of filaments forming an array is implanted within the tissue of a patient in need thereof so that the bioactive agent is released into the surrounding tissue. A plurality of the filaments can be disposed in a regular array within the tissue so that the bioactive agent released by the filaments is present throughout a volume of the tissue at a therapeutically effective concentration. When the array of filaments is associated with a base, the base and the array of filaments are adapted so that all the filaments are simultaneously inserted into the tissue, for example by application of pressure on the base sufficient to push the filaments into the tissue. Alternatively, a template can be used to insert filaments individually or in small sets, the template guiding the filaments into the proper location and orientation within the tissue.

The invention also provides a method for using a medical device of the invention in conjunction with convection enhanced delivery, wherein a solution is infused under a pressure head into the tissue wherein the medical device is implanted, either through the filaments or by means of a separate device.

The invention also provides a method for using a medical device of the invention in conjunction with other bioactive chemotherapeutic or radiotherapeutic agents administered by other routes, or in conjunction with other radiological methods of treatment.

### BRIEF DESCRIPTION OF THE DRAWING

FIG. 1 is a side view of an embodiment of the filament of the present invention.

FIGS. 2A and 2B show cross-sectional views of embodiments of the filament of FIG. 1.

FIGS. 3A, 3B, 3C, 3D, and 3E show a variety of tip designs of the filament of FIG. 1.

FIGS. 4A, 4B, 4C, 4D, 4E, 4F, and 4G show various optional tip-disposed anchors for the filament of FIG. 1.

FIGS. 5A, 5B, 5C, and 5D show various optional shaft-disposed anchors for the filament of FIG. 1.

FIG. 6 shows the filament of FIG. 1 comprising both tip anchors and shaft anchors.

FIG. 7 shows the filament of FIG. 1 further comprising ribs.

FIGS. 8A, 8B, and 8C show various arrays of a plurality of the filaments of FIG. 1 implanted in tissue.

FIG. 9 shows an array of the filaments of FIG. 1 affixed to a base.

FIG. 10 shows a plurality of conjoined matrices comprising bases each bearing the filaments of FIG. 1.

FIGS. 11A and 11B show matrices composed of a plurality of the filaments of FIG. 1 and a continuous base.

FIGS. 12A and 12B show matrices composed of a plurality of the filaments of FIG. 1 and a mesh or sieve base.

### DETAILED DESCRIPTION

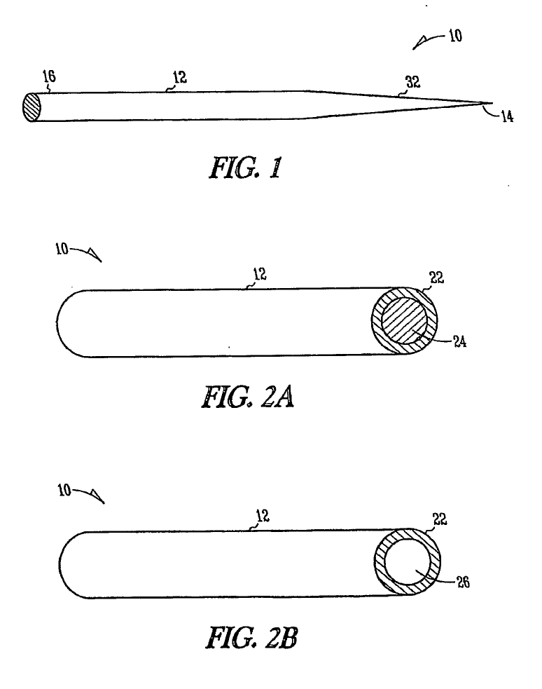

An embodiment of the present invention is directed to a medical device comprising an implantable filament, the filament being linear or curvilinear along its longitudinal axis. Referring to FIG. 1, a filament 10 of the invention can include a shaft portion 12, a tip portion 14, and a root portion 16. The filament is formed from a biocompatible material so that when it is implanted in the living tissue of a patient in need thereof, toxic or otherwise deleterious effects are minimized. Preferably, the filament is formed from a biodegradable material so that subsequent surgical removal after the bioactive agent is depleted is unnecessary, as the solid material is converted in situ into non-toxic, soluble breakdown products that are circulated away from the implantation site.

As used herein, the term “tip” refers to the end of a linear or curvilinear filament that is adapted to be the leading end of the filament when the filament is inserted into living tissue. The “shaft” refers to the portion of the filament between the tip and the trailing end when the filament is inserted into living tissue, which is termed the “root.” It is understood that in some embodiments according to the invention, the tip and the root may comprise different structures, whereas in other embodiments the tip and the root may be substantially identical, the distinction arising upon insertion of the filament into the tissue.

The filament is linear or curvilinear on its longitudinal axis to facilitate insertion into living tissue while minimizing the trauma or damage done by the operation of insertion. A linear or regularly curved filament can be inserted without creating a wound channel larger than the diameter of the filament. The tip of the filament is adapted to penetrate the tissue, preferably by gentle separation of the tissue and an absence of cutting action. The filament can be sufficiently flexible to allow for deflection of the filament if, in its passage through relatively soft tissue such as brain, it encounters a tougher structure such as a blood vessel. Thus, a filament such as that adapted to be implanted in brain tissue is preferably adapted to be of sufficient flexibility and deflectibility to minimize damage to blood vessels and the damaging hemorrhage that can result from their puncture. The tip and shaft in particular can be adapted to be free of cutting or puncturing edges or a point. A tip or a shaft of a filament can be adapted to minimize tissue trauma, for example by use of blunt tip shapes adapted to push tissue aside rather than cut through it or of smooth profiles with minimal tearing edges, or by suitable tapering of shaft diameter allowing for greater flexibility and deflectability of the section of the shaft adjacent to the tip relative to more rearward sections of the shaft. Various filaments can have differing physical properties adapted to be most favorable for insertion into a variety of tissue types and organs.

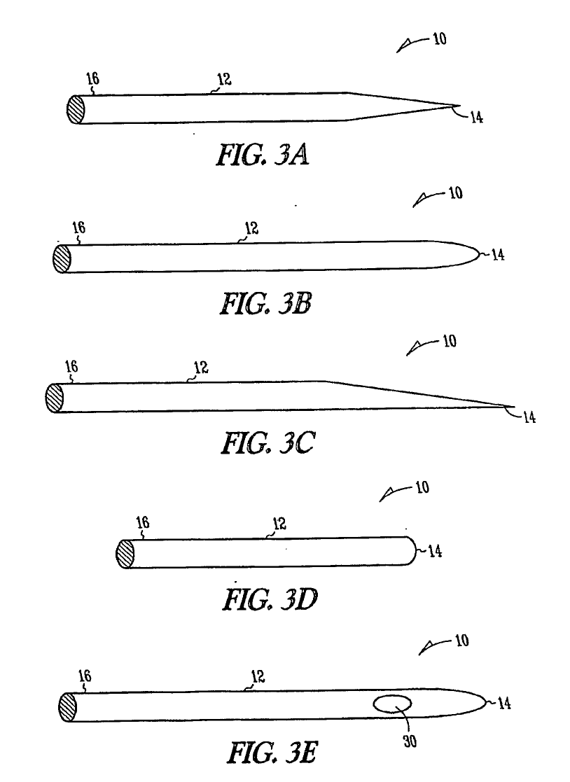

The filament is elongated, being of relatively small diameter in relation to its length. The filament can be of a diameter ranging from about 0.1 mm to about 5 mm, and of a length ranging from about 3 mm to about 100 mm. Preferably, the diameter of the filament is in the range of about 0.5 to about 2 mm, and the length of the filament is in the range of about 10 to about 30 mm. The filament can be straight throughout its length, or it can form an arc of constant radius. The shaft portion 12 of the filament can be of uniform diameter throughout its length, or it may comprise tapered sections, bulbous sections, and other deviations from uniformity. In cross-section the filament can be circular, as is shown in FIG. 2. However, the filament can also be of other cross-sections, such as polygonal, star-shaped, cross-shaped, ellipsoidal, trapezoidal, rhomboidal, or irregular cross-sections. The cross-sectional shape can be uniform throughout the length of the shaft, or it may change, for example, when anchors, ribs, or bulbs are present. The filament can be homogeneous in transverse cross-section, i.e., composed of a single material 20 without layering, or it can be layered as shown in FIG. 2(a), for example comprising an outer layer 22 and an inner layer 24. A layer may provide stiffening or reinforcement needed to achieve tissue penetration; for example an outer layer may be softer to minimize tissue damage and an inner layer harder to facilitate insertion, or, an outer layer may be adapted to contain high concentrations of a bioactive agent relative to an inner layer, which can itself be adapted to provide strength and elasticity. An inner layer can be hollow, providing a wall of sufficient rigidity to maintain a hollow core in an open state despite pressure from surrounding tissues, such as when it is desired to pump liquid material through the filament.

The filament may be porous, at least in part, comprising small voids which may be discrete or continuous. It can comprise more than two layers, of which one can be porous and the other solid, or any combination thereof. If there are multiple layers, the layers can be formed from different materials. Or, as is shown in FIG. 2(b), the filament can be hollow, comprising a single longitudinal hollow channel, with a wall 26 and a central void 28 as seen in cross-section. Alternatively, the filament can comprise multiple longitudinal hollow channels, interconnected with each other, or not.

The filament, as a whole, is of sufficient rigidity and strength to allow for implantation within at least soft solid tissue, such as within a brain tumor or surrounding brain tissue. It can be of a rigidity and strength to allow for implantation within tougher tissues, such as firm tumors or those with hardened surfaces as are characteristic of certain types of cancers located outside of the cranium, or into normal tissues of a firmer texture than central nervous system tissue. When two or more cross-sectional layers are present, at least one of the layers may provide additional rigidity for insertion, and the other layer or layers can contain the one or more bioactive agents. When more than a single layer is present, all the layers are biocompatible and preferably all the layers are biodegradable.

The tip 14 and root 16 of the filament are disposed at the opposite ends of the filament. The root is the trailing end of the filament when the filament is implanted within a patient's tissue, and the tip is the leading end. The root can be undifferentiated from the shaft, being a straight or curved cut terminating the filament and defining its length, or it can comprise a variety of structures. Or, the root may be of the same physical configuration as the tip. Alternatively, the root can comprise different physical structures from the tip. For example, the root can comprise a snap or a pressure fit device adapted to temporarily or permanently attach a filament to a base. The root can be permanently affixed to a base by other means, or may be contiguous with the base and provided as a single unit. The root can comprise a structural feature allowing for facile insertion of the filament into tissue or removal of the filament from tissue, for example a bead, rim, hole or flange. The root can comprise a structure wider than the diameter of the shaft, similar to the head of a nail. The root can comprise a structure adapted to allow the use of a specialized instrument for insertion of the filament into tissue. The root can comprise structures or features that allow the filament to be removed from tissue. For example, the root may comprise a small loop or flange that can be grasped with forceps. Alternatively, the root can comprise a small magnetic or a small piece of metal of a type that is attracted to a magnet, such that a magnet can exert sufficient force to extract a filament so modified from the tissue.

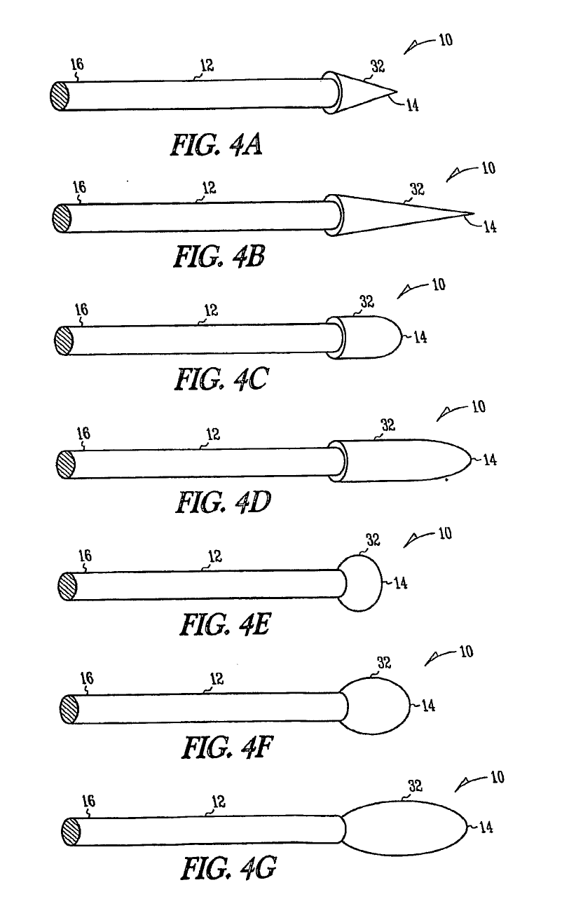

The tip 14 can take many forms, but is preferably adapted to minimize the degree of trauma to tissue upon insertion. Preferably the tip comprises a tapered structure, as is shown in FIG. 3. The tapered structure may be conical with a relatively sharp tip (FIG. 3(a)), or may be curved in, for example, a parabolic longitudinal cross-section (FIG. 3(b)), or may be beveled (FIG. 3(c)), or may be blunt (FIG. 3(d)). The tip may comprise additional features such as holes or pits 30 (FIG. 3(e)), such as can be used to allow dispersion of the contents of a hollow core or a small reservoir of liquid solution of a bioactive agent that is provided in addition to the bioactive agent contained in the biocompatible material that forms the filament. Alternatively a plurality of pits or holes can be disposed along the length of the shaft in order to increase the surface area of the filament in contact with tissue and fluids.

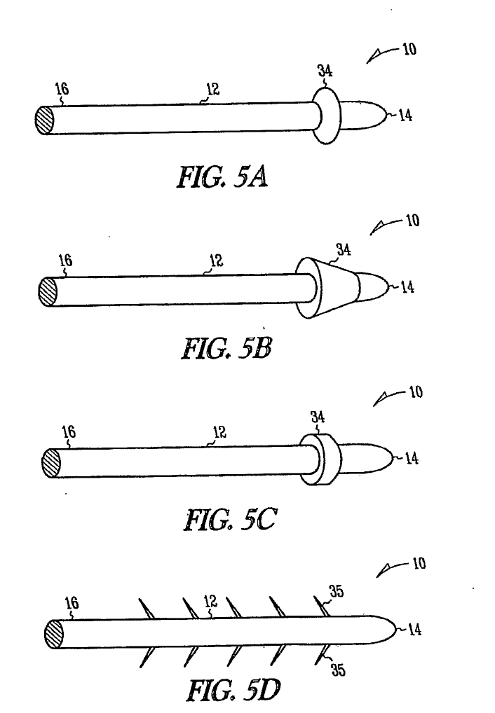

The tip 14 can also comprise a tip anchor 32. Referring to FIG. 4, a tip 14 can include a tip anchor having one of a variety of shapes. An anchor serves to retain the filament in position in the tissue in which it is emplaced, and can also serve to provide an additional volume of biocompatible material to contain the bioactive agent for release into the surrounding tissue. When the filament is inserted into the tissue, the tip anchor, which has at least in some portion of the anchor a diameter greater than that of the filament shaft 12, serves to anchor or immobilize the filament such that it resists expulsion from the tissue. The tip anchor can take the form of a barb (FIGS. 4(a)-(d)), with the trailing edge of the anchor tip presenting a substantially flat surface resisting withdrawal of the filament. The barbed tip anchor can be of a conical shape with a relatively sharp leading end (FIGS. 4(a), (b)), or can have a rounded or parabolic shape leading end (FIGS. 4(c), (d)). Alternatively, the tip anchor can be of a bulbous shape, wherein resistance to withdrawal of the tip is provided by a sloping or rounded trailing surface of the tip anchor (FIGS. 4(e)-(g)). The tip anchor is preferably adapted to minimize the degree of trauma that can result from insertion into the tissue.

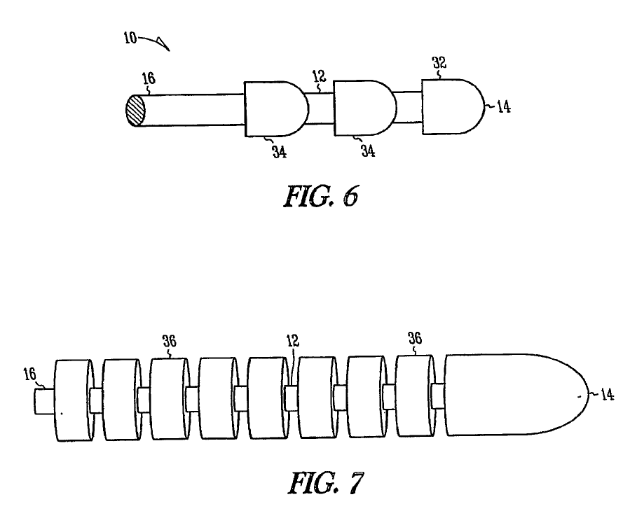

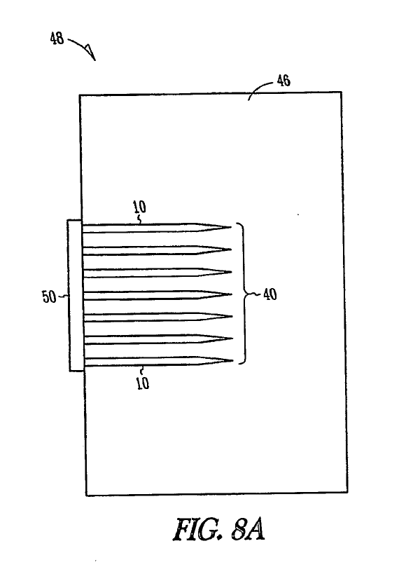

The shaft portion 12 of the filament 10 can also comprise a shaft anchor 34. Referring to FIG. 5, a shaft anchor is disposed on the shaft rearward of the tip. A shaft anchor may take the form of a barb, wherein a substantially flat surface facing rearward serves to resist withdrawal of the filament from the tissue (FIGS. 5(b), (c)). Alternatively, a shaft anchor may have a bulbous shape, where a sloping or curved surface resists rearward withdrawal of the filament from the tissue (FIG. 5(a)). The forward-facing surface of the shaft anchor may slope at a single angle with respect to the longitudinal axis (FIG. 5(b)) or may be curved (FIG. 5(c)). Alternatively, a shaft anchor can take the form of a hair barb 35, for example as shown in FIG. 5(d). One or a plurality of hair barbs can be disposed on the shaft. The hair barb can be of sufficient flexibility such that it lies flat against the shaft 12 when undergoing insertion into a tissue and of sufficient elasticity and rigidity that upon any rearward movement of the filament, the hair barb is displaced sideways to resist the movement. Hair barbs also can serve to increase the surface area of the shaft, which can serve to increase the rate of outflow of a bioactive agent when the outflow rate is limited by the size of the area of contact of the shaft and the surrounding tissue. Tip anchors and shaft anchors can both be provided on an embodiment of the inventive filament, for example as shown in FIG. 6.

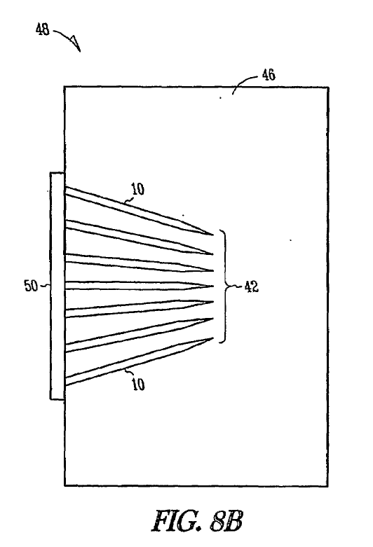

The shaft portion 12 can further comprise a plurality of ribs 36. Referring to FIG. 7, the ribs are disposed on the shaft 12 of the filament 10. The ribs can be all of the same size and shape, and are disposed in sufficiently close proximity to each other on the shaft to limit the amount of bending the shaft can undergo, while preserving a certain degree of flexibility. Preferably, the ribs are circular discs mounted concentrically on the shaft, such that the resistance provided for a given degree of tip deviation is uniform regardless of the direction of deviation. The rib discs can have chamfered edges in order to avoid the presence of a sharp edge that could cut or damage tissue. The ribs can be adapted to assist the filament in deflecting around obstacles the tip 14 of the filament encounters in the process of implantation into tissue. For example, as a filament is inserted into a relatively soft tissue, the rigidity of the filament is sufficient to keep the path substantially straight. However, it is possible that a less yielding inclusion may exist within the tissue, that would serve to deflect the filament in a markedly different direction such that when insertion is complete, the tip end of the filament may be in an entirely different location in the tissue than was anticipated. However, on a filament equipped with ribs 36, upon a definable degree of flexing of the shaft, the edges of the ribs come into contact and resist further deflection. Thus, the ribs serve to increase rigidity after a certain degree of deflection or bending of the filament has occurred. The ribs also increase the volume of the biocompatible material that can contain the bioactive agent, and the surface area of contact between the filament and the surrounding tissues.

The filament, including the shaft, tip, root, tip anchors, shaft anchors or ribs, can all be formed of a single biocompatible material, although various components or portions of the filament can also be composed of different materials. A preferred biocompatible material is also biodegradable. Preferred biocompatible, biodegradable materials include various types of organic polymers, particularly thermoplastic polymers. Suitable thermoplastic polymers for incorporation as the solid matrix of the controlled release polymer system are solids, pharmaceutically compatible and biodegradable by cellular action and/or by the action of body fluids. Examples of thermoplastic polymers include polylactides, polyglycolides, polylactide-glycolides, polycaprolactones, potyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene malonates, polyalkylene succinates, poly(malic acid) polymers, polymaleic anhydrides, poly(methylvinyl)ethers, poly(amino acids), chitin, chitosan, and copolymers, terpolymers, or combinations or mixtures of the above materials.

The material of which the filament is composed may further include plasticizers as may be needed to provide the necessary combination of rigidity, strength, flexibility, and smoothness. An example of a plasticizer is a phthalate ester. Another plasticizer is N-methylpyrrolidone.

Preferred polymeric materials are polylactide, polyglycolide, and poly(lactide-glycolide). These polyesters show excellent biocompatibility. They produce little, if any, tissue irritation, inflammation, necrosis, or toxicity. In the presence of water and enzymes present in living tissue, these polymers produce lactic and glycolic acids, respectively, which are not toxic and are readily metabolized by the body.

It is particularly advantageous when the entire filament is formed from a single biocompatible material, as the filament including the bioactive agent can be formed in a single operation by casting the polymer or its precursor in a suitable mold, optionally including suitable plasticizers or stabilizers. Alternatively, the filament can be pre-cast and the bioactive agent subsequently infused, as may be desirable when the bioactive agent is unstable under the conditions used to cast the polymer, or is a radioactive material with a short halflife.

The bioactive material can include any substance for which infusion into tissue is an appropriate therapeutic regimen. An example of a bioactive material is an anticancer agent, which may be a chemotherapeutic agent or a radiotherapeutic agent for which release into the tissue surrounding the implanted filament is desired.

Chemotherapeutic agents can include small molecule drugs such as anticancer drugs and prodrugs including alkylating agents, other types of anticancer drugs such as taxol or Vinca alkaloids, biological agents such as monoclonal antibody-coupled toxins, or apoptosis inducing agents, cell-cycle blocking agents, anti-angiogenesis agents, or any agent suitable for direct release within living tissue. A preferred example of a chemotherapeutic agent adapted to be released into tissue is carmustine (BCNU), an alkylating agent.

Radiotherapeutic agents can include radionuclides, optionally coupled to targeting agents, that are suitable for direct release into living tissues, for example, tumor-selective antitumor antibodies covalently linked to chelating moieties incorporating selected radionuclides. Example of radionuclides that may be provided, optionally in chelated form, include ³²P, ⁶²Cu, ⁶⁴Cu, ⁷⁷ Br, ^{80m}Br, ⁹⁰Y, ⁹⁷Ru, ¹⁰⁵Rh, ¹⁰³Pd, ¹⁰⁹Pd, ¹¹¹In, ¹²³I, ¹²⁴I, ¹³¹I, ¹⁵³Sm, ¹⁶⁶Ho, ¹⁸⁶Re, ¹⁸⁸Re, ¹⁹²Ir, ¹⁹⁸Au, ¹⁹⁹Au, ²⁰³Pb, ²¹¹Pb, ²¹¹At, or ²¹³Bi. Other suitable radionuclide-containing molecular entities include S-phase specific radiotoxic nucleosides, such as uridinedeoxynucleoside incorporating radionuclides such as ¹²³I, ¹²⁴I, ¹²⁵I, ¹³¹I, ⁷⁷Br, ^{80m}Br, ²¹¹At, or ²¹³Bi. A specific example of a radiotherapeutic agent adapted to be released into tissue is ¹²³I or ¹²⁵I-iodouridinedeoxyriboside (IUDR), an Auger electron emitter, that is phosphorylated in vivo and is then incorporated into replicating cellular DNA. Another preferred example of a radiotherapeutic agent is ¹²³I- or ¹²⁵I-IUDR covalently bonded, for example by phosphate and carbonyl esters, to a high molecular weight substance such as dextran, clearance from the tissue of which is slower than that of IUDR in free form, but which hydrolyzes over time to provide IUDR in free form so that it may be phosphorylated to provide the corresponding nucleotide, which is then incorporated into newly synthesized DNA that resides in the nucleus of a cell. In that situation, the IUDR is well-situated to kill the cell with minimal damage to surrounding cells, through the emission of short-range Auger electrons.

A filament of the invention can also comprise additional components, such as a radiopaque agent to allow visualization by fluoroscopy, or a radioactive agent suitable for imaging using techniques such as SPECT or PET, or an MRI-active agent to enhance visualization by magnetic resonance imaging. An example of a radiopaque agent is iohexyl, an organoiodine compound. An example of a radioactive agent is a mixture of ¹²³I- and ¹²⁴I-labelled materials, optionally in combination with a ¹²⁵I-labelled materials, preferably in relative proportions of radioactivity of about 1:1:8. Such a mixture emits gamma photons suitable for detection, such as with a SPECT apparatus. An example of a MRI-active material to enhance visualization by magnetic resonance imaging is gadolinium.

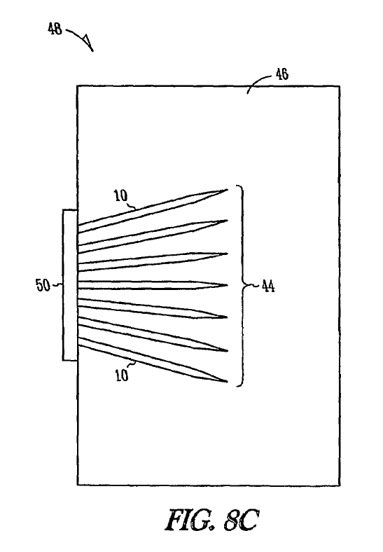

The invention further provides an array of the filaments implanted in tissue, such as in tumor tissue or in normal tissue surrounding a tumor locus. As the term is used herein, an “array” refers to a plurality of filaments of the invention implanted in tissue in a defined spatial configuration. Referring to FIGS. 8(a)-(c), examples of three arrangements of arrays, 40, 42, and 44 of filaments 10, disposed within brain tissue 46 adjacent to a site of an excised tumor 48, are shown. The filaments making up the array are preferably disposed in a regular manner, which may be altered according to the medical needs of the given situation. An array can be held in a particular spatial configuration by means of a base 50. Or, an array can be created in situ through the sequential implantation of filaments or sets of filaments using an array template. For example, array 40 is a parallel array comprising a plurality of the filaments 10. Each filament in the parallel array is aligned parallel to every other filament. Preferably, the filaments are spaced apart no more than about twice the distance that the therapeutic agent they contain would be expected to diffuse into the tissue and be present in the tissue at effective concentrations. Thus, if an agent is expected to have an effective diffusion radius from the filament of about 5 mm, the filaments are preferably spaced no more than about 10 mm apart. Thus, a parallel array is effective in maintaining a relatively uniforn concentration of the therapeutic agent throughout a defined volume and region of target tissue.

In another example, a negative radial array 42 comprises filaments 10 disposed within the tissue such that the shafts of the filaments converge towards the tips, and the tips are in relatively close proximity to each other. An array of this type can be used, for example, when a physician may have reason to believe that it would be medically indicated to achieve a particularly high concentration of the therapeutic agent at a point within the tissue. By aiming the filaments such that they “focus” on a particular region, it is expected that a concentration gradient of the agent will be formed where the concentration is highest in the vicinity of the tips and adjacent portions of the shaft where they are disposed relatively close together within the tissue.

In another example, a positive radial array 44 comprises filaments 10 disposed within the tissue such that the shafts deviate away from each other at a fixed angle. An array of this type can be used, for example, to infuse a therapeutic agent into tissue adjacent to a curved surface of a void resulting from excision of a tumor. It would be expected that, absent convection or fluid flow, the concentration profile of the released agent will form a gradient, higher near the roots of the filaments and lower near their tips. This could be advantageous in treatment, for example, of GBM where, as was discussed above, the majority of tumor recurrence takes place within 2 cm of the site of the excised tumor with lower rates as the distance from the tumor site increases. When the positive radial array 44 of filaments is emplaced from the void created by removal of a tumor when a negatively curved surface remains, regions of low concentration of the released agent in close proximity to the site of the excised tumor are avoided. Thus, the tissue closest to the void receives the highest, consistent dose of the therapeutic agent with a minimum of regions receiving little agent. Again, it is preferred that the filaments be emplaced close enough to each other that they are, on the average, no further apart than twice the effective diffusion range of the agent for at least most of their length.

Different types and designs of the inventive filaments can be used within an array emplaced within tissue. The filaments need not be all of the same type, shape, or configuration. For example, filaments can be of various lengths, such as to accommodate local anatomical structures, or be of differing diameters, or have different shaft or tip anchor types.

In an embodiment of the invention, an array template can be used to create a spatially defined array of filaments within the target tissue. An array template is a structure that serves to guide each filament separately, or pluralities of filaments in sets, into their target position within the tissue. The device including the array template and the plurality of filaments adapted to be guided by the template is referred to herein as a “array assembler.” An array tempate is adapted to direct the filaments through physical contact between the template structure and the shafts of the filaments as the filaments are inserted through the guiding structures of the template. For example, an array template can be a multi-holed plate, wherein each hole serves to guide each respective filament inserted therethrough into a defined position within the target tissue, so that the spatially defined array is obtained. Each hole is adapted to provide sufficient guidance to its respective filament to firmly guide the filament into position against at least some tissue resistance. The template need not be adapted to remain in the tissue following emplacement, and can be removed prior to closure of the surgical incision, but it can be left in place, particularly if it is formed of a biodegradable material. Thus, the inventive array assembler is a device adapted to form an inventive filament array within the target tissue of a patient.

An array as formed through the use of a template can include parallel filaments, or radially arrayed filaments. As the filaments are inserted into the tissue individually or in small groups using the array template, they can be directed in various directions into the tissue without causing tissue tearing. For example, a template can provide directionality to the insertional motion of each filament such that a radial array, either positive or negative, can be formed. The template itself can be substantially flat, or can be curved positively or negatively, or can be of an irregular shape, provided that each of the openings through which each of the plurality of filaments passes is angled to yield the desired final configuration of the array.

For example, referring to FIG. 13, the filaments can be guided into position by means of openings in the template through which the filaments are inserted, the openings being adapted to provide sufficient support to a portion of the filament shaft to align it in the desired direction for insertion. In a radially-arranged matrix according to this embodiment, each individual filament being straight or curvilinear can be inserted without causing tearing or a wound channel larger than the diameter of the filament. Once inserted, the filaments may become affixed to the template, for example by a snap or a pressure fit. Alternatively, once the filaments are emplaced using the template, the template can be removed to provide, for instance, the radially disposed array of filaments within the tissue as described above.

It is understood that a combination of such arrangements of arrays can be used in a given medical situation. Furthermore, additional single filaments or irregular arrays can be used as medically indicated.

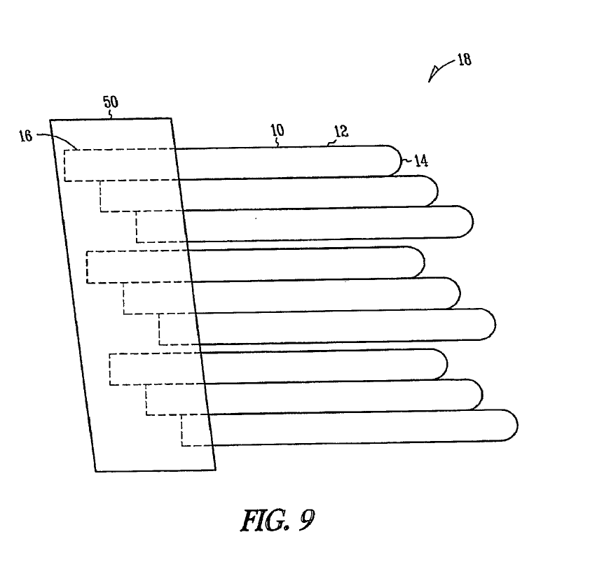

An embodiment of the invention further provides a matrix of a plurality of filaments 10 and a base 50. Referring to FIG. 9, the filaments may be reversibly associated with or permanently affixed to the base to provide a matrix. The base holds the array of filaments in a defined pattern. The base is adapted to receive the filaments and secure them by their roots 16, so that the base secures the filaments and guides the filaments into position when the filaments are emplaced within a tissue. Thus the base is adapted to define both the pattern of the points of attachment of the filaments to the base, and the direction each filament projects from the base. By doing so, the base serves to define the spatial configuration of the array of filaments disposed within the tissue after emplacement, which can be achieved by applying slight pressure to the base as the tips of the filaments are disposed at their desired entry positions on the surface of the target tissue.

The matrix can comprise a base with filaments that are permanently affixed, or filaments from which the base can be removed following insertion of the filaments into the tissue. The base with the filaments is surgically emplaced as a unit, as opposed to the use of an array template, wherein the filaments are individually or in subsets emplaced using the array template as a guide. The roots of the filaments make contacts with the base that serves to temporarily or permanently secure the filaments to the base and to provide directional guidance to the filaments during insertion. The base may be adapted to be removed from the filaments after they are inserted into the tissue to form the array, prior to closure of the surgical incision, or the base may be adapted to remain affixed to the array of inserted filaments and remain within the tissue after surgery. The configuration of the matrix is defined by the pattern of disposition of the filament roots on the base and by the orientation of the filaments with respect to one another. Thus, in an embodiment of the present invention comprising a matrix, the configuration of the matrix defines the configuration of the array that is created within the tissue upon emplacement of the matrix therein.

The filaments may be disposed on the base at any suitable spatial density. Thus, for a base of a given size, the number of filaments associated with the base can vary depending on the density of filaments needed for a particular application. Preferably, the filaments at their roots are separated from each other on the base by no more than about 10 mm when the matrix is used for implantation within central nervous system tissue, due to the known distance limitations of the diffusion of substances within this type of tissue. However, for other applications, the distribution of the filaments on the base may be less dense. The roots of the filaments may form a rectilinear arrangement on the base, or may form staggered rows, or may be arranged in concentric circles, or may have a distribution of density on the base that varies with the position on the base; for example, close together at one side of the base and further apart on the other with a gradient distribution between them.

The filaments and the base are adapted to be inserted into tissue as a unit; for example by applying pressure to the base, the array of filaments is simultaneously emplaced within the tissue while the base rests upon the surface of the tissue. The base itself is not adapted to penetrate the tissue to any significant degree. In this embodiment, the filaments are preferably disposed in a parallel array in order to minimize any tissue tearing that could result if the filaments were other than parallel to each other.

The base may be of any suitable size; a larger base may comprise many filaments, and a smaller base typically fewer, in order to allow the physician to choose the size and arrangement most suitable for the particular situation confronting the physician. For example, it may be advantageous to emplace a plurality of small base units when one tissue configuration exists, or a single larger base unit in the case of another tissue configuration. Typically, a base can be of the dimensions of about a centimeter ranging up to several centimeters across.

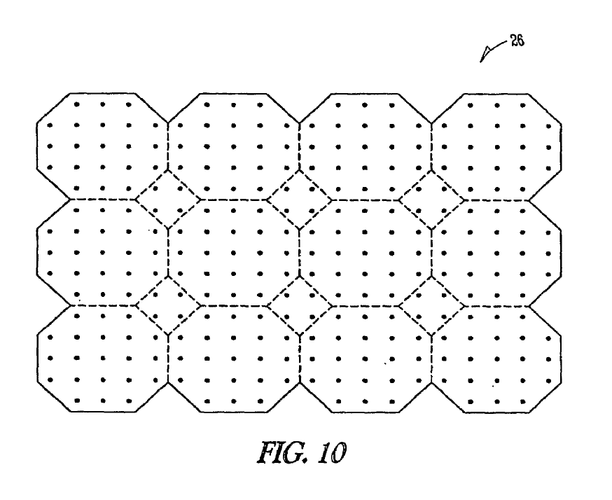

The base can be of any suitable shape, such as square, rectangular, circular, ellipsoidal, hexagonal or a custom form adapted to fit a specific shape need defined by the physician for a particular medical situation or tissue configuration. It can be flat, curved, or irregularly shaped, as desired for a given medical situation. A base may comprise features along the edge that allow it to be coupled or attached to adjacent base units, so that the matrices comprising the bases and their associated filaments are conjoined. Referring to FIG. 10, a set of matrices with octagonal bases 52 and square bases 54 are attached by joints 56 at the edges. The joints 56 between the bases may be bendable, such that a plurality of matrices may be inserted into tissue concurrently, the bendable joints allowing the plurality of matrices to better conform to irregularities in the surface of the tissue to which the matrices are applied. Alternatively, the joints may be separable, such that the physician can remove units from or attach units to a set of conjoined matrices as may be needed. In another alternative, the joints may be formed by attachment of individual matrices whose base edges are provided with features allowing them to couple and form the joint. This attachment may be reversible or irreversible.

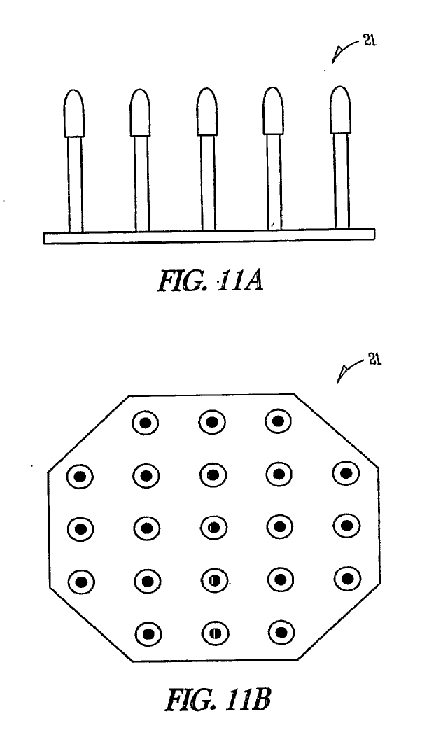

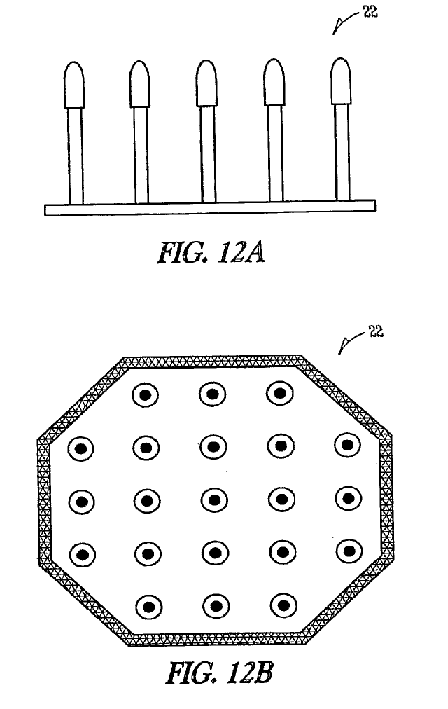

The base can be continuous with no openings in it (see FIG. 11), or it can comprise a mesh or sieve through which fluids can pass (see FIG. 12). In either case the base is of sufficient strength and rigidity such that it can be handled and emplaced on the tissue. The base can be of any suitable thickness; typically on the order of a few millimeters or less in order to provide sufficient support and guidance for the filaments when they are emplaced into the tissue. The base can be flat, or can be curved or folded to fit the needs of the particular tissue configuration or therapeutic situation. For example, in the formation of a negative radial array, the base is can curved to fit the surface of the tissue on which it will be emplaced. A negative radial array can be created by emplacement of filaments in a flat base wherein the guiding features point the filaments in the correct direction for emplacement, but when the surface of the tissue is positively curved, use of a curved base may be indicated.

A base can further comprise features to assist in the removal of the base or the matrix from tissue on or in which it has been emplaced. For example, a loop, graspable by forceps, can be disposed on the side of a base opposite the side on which the filaments are disposed, such that a surgeon can grasp it with a suitable instrument. Alternatively, the base may comprise a metal which is capable of responding to a magnet.

An embodiment of the invention provides a method of treating a malcondition, comprising emplacing one or more of the implantable filaments into tissue in of a patient in need thereof, so as, in the case of use of a plurality of filaments, to create an array of the filaments in the tissue. An array template can optionally be used. Or, a method can comprise implanting one or more matrices into the tissue of the patient to create an array of the filaments in the tissue. The filaments are of sufficient strength and rigidity to undergo implantation into the target tissue. The term “target tissue” as used herein refers to living tissue of a patient wherein a malcondition exists that the filaments containing the bioactive agent are adapted to treat, and into which the filament, an array of the filaments, or a matrix comprising a plurality of the filaments, are inserted as a method of treatment. For example, central nervous system tissue surrounding the site of an excised GBM constitutes target tissue when implantation of filaments comprising an appropriate chemotherapeutic or radiotherapeutic substance into that tissue is medically indicated.

An embodiment of the inventive method comprises emplacement of a filament or a plurality of filaments within the target tissue such that the therapeutic substance contained within the biocompatible material of the filament is released over a period of time into the surrounding tissue. Preferably, an array of the filaments in the target tissue is created through emplacement of a plurality of the filaments, optionally further comprising a base, into the tissue. For example, after surgical excision of a tumor from central nervous system tissue, as in removal of a localized GBM tumor of advanced stage, a void is formed with walls consisting of the surrounding brain tissue. An embodiment of the method of the invention comprises creating an array of the filaments within this surrounding brain tissue, wherein the filaments are spaced in a regular array reaching to a defined depth in the tissue in such areas of the void walls as is deemed medically indicated by the physician.

More than a single bioactive agent may be used in any of the embodiments of the invention. For example, an array of filaments when emplaced in tissue may release both a radiotherapeutic agent and a chemotherapeutic agent, or a plurality of radiotherapeutic agents or of chemotherapeutic agents or any combination thereof.

A type of chemotherapeutic agent that a filament can include is an alkylating agent. An alkylating agent is believed to act by alkylation of DNA, which interferes with DNA replication and is thus most toxic for cells undergoing replication. Examples of alkylating agents include carmustine and other nitrosoureas, nitrogen mustards such as cyclophosphamide or chlorambucil, triazines such as temozolomide, sulfonate esters such as busulfan, and the like. Or, a chemotherapeutic agent can be another type of anticancer agent, such as taxol or a Vinca alkaloid. Alternatively a chemotherapeutic agent can be a monoclonal antibody-coupled toxins, an apoptosis inducing agent, a cell-cycle blocking agent, an anti-angiogenesis agent, or any other type of small molecule or macromolecular agent that can provide a beneficial effect in causing remission of the tumor, slowing the tumor's growth, inhibiting tumor metastatis, or otherwise prolonging the patient's survival.

A type of radioactive agent that a filament can comprise is an Auger electron emitter. The Auger-emitting radionuclide is incorporated into a chemical entity that is adapted for uptake into target cells, in which case the short-range Auger electrons exert their destructive effects directly on the cell in which they are contained with minimal collateral damage to surrounding cells. A specific example of such a radiochemical entity is ¹²³ or ¹²⁵I-iodouridinedeoxyriboside (IUDR). Other Auger electron emitting isotopes can also be used, for example, incorporated into molecular entities that target chromosomes either covalently or through use of chelating moieties.

In one embodiment of the inventive method, a matrix comprising a base to which a plurality of filaments are permanently affixed is inserted into the wall of the void substantially immediately after removal of the tumor. The matrix, charged with a bioactive material medically indicated for treatment of the tumor, is pushed into the surrounding tissue at a location decided by the physician supervising the operation. The matrix is inserted in the tissue in such a way as to minimize trauma, puncture of blood vessels, and destruction of healthy neurons within the tissue. The specific details of the matrix, such as the size and spacing of the filaments, the geometry of the array, the identity and quantity of the bioactive agent, the controlled release properties of the biocompatible, preferably biodegradable material of which the filaments are composed, and other vital factors are decided by the physician based on knowledge and experience. The matrix configuration selected for a particular patient is adapted to meet the medical needs of the that patient. Preferably, a parallel array is created in order to minimize the wound channels created by inserting the plurality of filaments simultaneously by application of gentle pressure to the base on the side opposite the filaments. Optional anchors on the tip and shaft of some or all of the filaments in the array assist in securing the device in position within the tumor, resisting displacement of the filaments once the array is formed. If the filaments used are biodegradable, the base preferably is also biodegradable so that no second surgical procedure is necessary for removal of the base.

In another embodiment, the base is removable following emplacement of the array of filaments during the initial surgical procedure, leaving the array of filaments disposed within the tissue. In this embodiment, the base need not be biodegradable. The base may be detached from the inserted array of filaments by any suitable procedure. For example, the base may be adapted to release the roots of the filaments if lateral pressure is applied, or sockets in the base in which the filament roots are held for insertion into tissue may be shaped such that resistance to movement of the base away from the roots is minimally resisted, thus releasing the filaments without disturbing their emplacement.

In another embodiment of the inventive method, an array template is emplaced on the area of the void wall resulting from tumor removal, and filaments are inserted through the guiding features of the template individually or in small sets to form the array within the tissue. In this embodiment, there is no requirement that the array that is created in the tissue is a parallel array. As the filaments are not all inserted simultaneously, this embodiment can be adapted for creation of radial arrays in the target tissue. Filaments are inserted through the guiding features of the template such that tearing of the tissue is minimized and the wound channel created by a filament is no greater than the diameter of the filament. The template can be adapted to remain associated with the array of filaments following emplacement, in which case the base is preferably biodegradable if the filaments are biodegradable. Alternatively, the base can be adapted to be removed following emplacement.

In another embodiment of a method of the invention, no base is used in creating the array of filaments within the target tissue. Using any suitable means of handling the filaments, the filaments are inserted by hand or hand tool into the target tissue. In this embodiment, the surgeon has a high degree of flexibility in terms of the array that is created by insertion of the plurality of filaments. Another embodiment of the present invention comprises the use of convection enhanced delivery in conjunction with a filament, an array of filaments, or a matrix comprising a plurality of filaments of the invention. As the term is used herein, “convection enhanced delivery” refers to a high-flow microinfusion delivery technique that assists and enhances the dispersion of an substance such as a chemotherapeutic compound that is introduced into brain tissue. For example, refer to Walter A. Hall, M.D., Edward Rustamzadeh, M.D. and Anthony L. Asher, M.D., “Convection-enhanced delivery in clinical trials,” Neurosurg Focus, 14(2), 1-4, (2003), incorporated herein by reference. Convection-enhanced delivery serves to create fluid flows within brain tissue that can disperse an introduced material further than the material would be expected to penetrate by diffusion alone.

In a method according to the present invention, convection-enhanced delivery is used in conjunction with a method of the invention for dispersion of a therapeutic agent released from a filament or filaments of the invention. The bioactive agent, released by the filaments of the invention, is further distributed and dispersed into adjacent tissue by introduction of a fluid under sufficient pressure to produce the convection-enhanced dispersal of the agent. The fluid may be introduced by any suitable means, such as are disclosed in Bobo, R H, Laske D W, Akbasak A et al., “Convection-enhanced delivery of macromolecules in the brain,” Proc Natl Acad Sci USA, 91:2076-2080, (1994), which is incorporated herein by reference. For example, a suitable fluid such as saline may be introduced under pressure into target tissue after emplacement of an array of the filaments of the invention. The fluid flow induced by introduction of the saline serves to aid in the dispersal of the bioactive agent throughout the target tissue, extending the distance from the filaments where a therapeutically effective dose of the bioactive agent can be achieved.

The fluid flow of the convection enhanced delivery can be provided by a filament or a plurality of the inventive filaments. The pressurized fluid can be transmitted through a filament or a plurality of filaments such that the liquid emanates from the filaments within the tissue, providing the convection enhanced delivery of the bioactive agent released by the filaments. The same filaments that deliver the bioactive substance can also provide the fluid flow, or alternatively, different subsets of filaments, optionally of different configurations, can deliver the bioactive agent and provide the fluid flow respectively. Thus the convection enhanced delivery fluid flow is outwards from each fluid-delivering filament, and the total pressure gradient thus created would be expected to drive the bioactive agent deeper into the tissue as well as laterally. Alternatively, the fluid flow can take place through the base, such that a pressure gradient is created from the base towards the tips of the filaments, thus driving the bioactive agent further into the tissue.

A further embodiment of a method of the present invention for treating a malcondition further comprises combination therapy wherein a filament, an array of filaments, or a matrix comprising filaments of the invention is emplaced within tissue in conjunction with administration of a second therapeutic substance adapted to treat the malcondition, or with administration of radiation of a second type such as MeV external beam radiation or brachytherapy. For example, in the embodiment wherein an Auger electron emitting radionuclide incorporated into a nucleoside analog is the bioactive agent released by the filaments, a second type of radiation such as, for example, beta-particles or gamma-rays may also be provided, such as from an external source or by brachytheraphy.

Alternatively, in the embodiment where ¹²³I or ¹²⁵I-IUDR is a radiotherapeutic agent released from the array of filaments disposed within the target tissue, a chemotherapeutic agent may be administered by another route. Chemotherapeutic agents can include small molecule drugs such as anticancer drugs and prodrugs including alkylating agents, biological agents such as monoclonal antibody-coupled toxins, or apoptosis inducing agents, cell-cycle blocking agents, anti-angiogenesis agents, or any agent suitable for direct release within living tissue. A specific example is oral administration of temozolomide, a prodrug for an alkylating agent type anticancer medicament.

In another embodiment, one chemotherapeutic agent can be released from the filament or array filaments, and a second chemotherapeutic agent can be provided via another route. For example, carmustine can be provided from a filament, array, or matrix of the invention, and temozolomide can be administered orally. Or, a chemotherapeutic agent can be provided to the target tissue of the patient by the filament, array or matrix of the invention and radiation such as MeV external beam radiation, or radiation from implanted, sealed radioactive sources can also be provided.

It is to be understood that while the invention has been described in conjunction with the detailed description thereof, the foregoing description is intended to illustrate and not limit the scope of the invention, which is defined by the scope of the claims. Other aspects, advantages, and modifications are within the scope of the claims and will doubtless be apparent to persons of ordinary skill in the art.

1-59. (canceled)

60. An implantable filament adapted for release of a bioactive agent within a tissue of a living organism for treatment of a malcondition in a patient in need thereof, the filament comprising a biocompatible material and the bioactive agent, the filament being linear or curvilinear in form, having a tip, a shaft, and a root, and being of sufficient strength and rigidity to enable the filament to be inserted into the tissue, the bioactive agent or agents being releasably disposed within the filament so that upon implantation the bioactive agent disperses into the tissue.

61. A filament array, comprising an array of the filaments of claim 60 disposed in a target tissue of a patient in need thereof.

62. An array assembler comprising a plurality of the filaments of claim 60 and an array template, the template being adapted to guide each respective filament into a position within target tissue of a patient so as to form the array of claim 61 in the target tissue.

63. A matrix, the matrix comprising a plurality of the filaments of claim 60, and a base, the base comprising a mounting structure for the filaments such that the filaments are held in a defined spatial relationship when affixed thereto, the filaments being mounted on the base on a first side thereof and projecting therefrom, the base being adapted to support each filament with sufficient rigidity to enable the filaments to be inserted into a target tissue of a patient in need thereof to form the array of claim 61.

64. The filament of claim 60 or the filament array of claim 61 wherein the malcondition comprises a neoplasm.

65. The filament or the filament array of claim 64, wherein the neoplasm comprises an advanced stage localized solid tumor.

66. The filament or the filament array of claim 64, wherein the neoplasm comprises a malignant glioma.

67. The filament or the filament array of claim 64, wherein the malignant glioma comprises glioblastoma multiforme, anaplastic astrocytoma, or anaplastic oligodendroglioma.

68. The filament of claim 60, wherein the filament is biodegradable.

69. The filament of claim 60, wherein the tissue is central nervous system tissue.

70. The filament of claim 60, wherein the bioactive agent is dispersed throughout the filament.

71. The filament of claim 60, wherein the bioactive agent comprises a pharmacological agent.

72. The filament of claim 60, wherein the bioactive agent comprises a radiological agent.

73. The filament of claim 72 wherein the bioactive agent comprises a radiolabelled nucleoside or nucleoside analog, ¹²³I- or ¹²⁵I-IUDR; ¹²³I, ¹²⁵I, ²¹¹At, ²¹³Bi, ^{80m}Br, ¹²⁴I, or a ⁷⁷Br-labelled nucleoside analog, or prodrugs thereof.

74. The filament of claim 60, comprising more than one cross-sectional layer.

75. The filament of claim 60, having a uniform cross-section throughout the length of the shaft.

76. The filament of claim 60, having a circular, polygonal, cross-shaped, or star-shaped cross-section.

77. The filament of claim 60, the shape of the tip and the shape of the shaft thereof being adapted to minimize trauma to the tissue into which the filament is implanted.

78. The filament of claim 60, further comprising one or more anchors disposed on the shaft.

79. The filament of claim 60, wherein the filament is about 0.1 to about 5 mm in diameter.

80. The filament of claim 60, wherein the filament is about 0.5 to about 2 mm in diameter.

81. The filament of claim 60, wherein the filament is about 3 to about 100 mm in length.

82. The filament of claim 60, wherein the filament is about 10 to about 30 mm in length.

83. The filament of claim 60, wherein the biocompatible material comprises an organic polymer.

84. The filament of claim 83, wherein the organic polymer comprises polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene malonates, polyalkylene succinates, poly(malic acid) polymers, polymaleic anhydrides, poly(methylvinyl)ethers, poly(amino acids), chitin, chitosan, and copolymers, terpolymers, or combinations or mixtures of the above materials.

85. The filament of claim 60, comprising ribs.

86. The filament of claim 60, wherein the root comprises a feature adapted to allow withdrawal of the filament from tissue in which the filament is disposed.

87. The filament array of claim 61, wherein the filaments are disposed in a parallel arrangement.

88. The filament array of claim 61, wherein the filaments are disposed in a radial arrangement.

89. The array assembler of claim 62, wherein the template is adapted to dispose a plurality of the filaments within the target tissue in a parallel array.

90. The array assembler of claim 62, wherein the template is adapted to dispose a plurality of the filaments within the target tissue in a radial array.

91. The array assembler of claim 62, wherein the template is biodegradable.

92. The array assembler of claim 62, wherein holes in the template adapted to guide the filaments into the tissue are disposed thereon in a regular rectilinear pattern.

93. The array assembler of claim 62, wherein holes in the template adapted to guide the filaments into the tissue are disposed thereon in a regular concentric circular pattern.

94. The matrix of claim 63, wherein the base holds the filaments in a parallel array.

95. The matrix of claim 63, wherein the roots of the filaments are detachably affixed to the base.

96. The matrix of claim 63, wherein the roots of the filaments are permanently affixed to the base.

97. The matrix of claim 63, wherein the filaments are disposed on the base such that the roots thereof comprise a regular rectilinear matrix on the base.

98. The matrix of claim 63, wherein the filaments are disposed on the base such that the roots thereof comprise a regular concentric circular array on the base.

99. The matrix of claim 63, wherein the base and the filaments are biodegradable or bioerodable.

100. The matrix of claim 63, wherein the base is removable from the filaments after insertion of the filaments into the tissue.

101. The matrix of claim 63, wherein the base is substantially flat in form.

102. The matrix of claim 63, wherein the base is curved or folded in form.

103. The filament of claim 60 further comprising a radiopaque agent, a radioactive material for visualization, an MRI-active agent to enhance magnetic resonance imaging, or any combination thereof.

104. A method of treating a malcondition, comprising emplacing one or more of the filaments of claim 60, or one or more the filament arrays of claim 61, or one or more matrices of claim 63, into a target tissue of a patient in need thereof.

105. The method of claim 104, wherein the malcondition comprises neoplasia.

106. The method of claim 104, wherein the malcondition comprises a solid tumor.

107. The method of claim 104, wherein the malcondition comprises an advanced stage localized solid tumor.

108. The method of claim 104, wherein the tissue comprises central nervous system tissue.

109. The method of claim 104, wherein the target tissue comprises central nervous system tissue surrounding a void resulting from resection of a central nervous system tumor.

110. The method of claim 109, further comprising fluid flow of convection enhanced delivery of the bioactive agent.

111. The method of claim 110, wherein the fluid flow comprises infusion of saline into the tissue.

112. The method of claim 110, wherein the matrix is adapted to provide the fluid flow of the convection enhanced delivery.

113. The method of claim 110, wherein the fluid flow is infused via the base or via the filaments.

114. The method of claim 104, further comprising administration to the patient of a second bioactive agent or an ionizing radiation from a second source.

115. The method of claim 114, wherein the route of administration of the second bioactive agent is directly into the tissue, is directly into the tissue with convection enhanced delivery, or is via the circulatory system.

116. The method of claim 114, wherein the radiation is ionizing radiation administered by external beam megavoltage radiation or by brachytherapy.

117. The method of claim 114 wherein the second bioactive agent is administered orally or via the circulatory system.

118. The method of claim 117 wherein the second bioactive agent is temozolomide and the administration is oral.
